(19) United States
(12) Patent Application Publication
(10) Pub. No.: US 2013/0204341 A1
(43) Pub. Date: Aug. 8, 2013

(54) METHOD AND APPARATUS FOR SELECTIVELY ACTIVATING CELLS BY APPLYING ELECTRICAL SIGNALS IN A SPATIAL PATTERN

(71) Applicants: Eduardo-Jose Chichilnisky, Cardiff, CA (US); Lauren Jepson, San Diego, CA (US); Pawel Hottowy, Gliwice (PL); Wladyslaw Dabrowski, Krakow (PL); Alan M. Litke, Ferney-Voltaire (FR)

(72) Inventors: Eduardo-Jose Chichilnisky, Cardiff, CA (US); Lauren Jepson, San Diego, CA (US); Pawel Hottowy, Gliwice (PL); Wladyslaw Dabrowski, Krakow (PL); Alan M. Litke, Ferney-Voltaire (FR)

(21) Appl. No.: 13/809,119

(22) PCT Filed: Jul. 12, 2011
(86) PCT No.: PCT/US2011/043707

§ 371 Date: Mar. 25, 2013

Related U.S. Application Data
(60) Provisional application No. 61/363,552, filed on Jul. 12, 2010.

(51) Int. Cl. A61N 1/05

(52) U.S. Cl. CPC A61N 1/0543; A61N 1/0541
USPC 607/137; 607/116

(57) ABSTRACT

A method and apparatus for applying electrical signals in specific spatial patterns to selectively activate sensory cells. The method comprises interfacing a population of cells with two or more electrodes, identifying target cells within the population to activate by electrical signals; and applying electrical signals in a spatial pattern to the population via the electrodes, such that the target cells are selectively activated in the desired spatial pattern.

### FIELD OF THE INVENTION

The present disclosure relates to methods, systems, and apparatuses for applying electrical signals in specific spatial patterns to selectively activate cells in order to develop interfaces to the nervous system.

### DISCUSSION OF RELATED ART

Sensory neural prostheses are devices that can substitute for a sensory modality that may have been damaged by injury or disease, as, for example, when the damage renders sensory cells incapable of accurately transmitting the different aspects of a sensory stimulus, which may be complex, to the brain.

### SUMMARY OF THE INVENTION

Disclosed is a design for the function of a neural prostheses capable of activating sensory cells in specific patterns in order to capture the complexity of the stimulus and replace the lost of function of a sensory modality. In one aspect, the disclosure refers to a method comprising interfacing a population of cells with two or more electrodes; identifying target cells within the population to activate by electrical signals; and applying electrical signals in a spatial pattern to the population via the electrodes, wherein the target cells are activated. The population of cells can include sensory cells, such as retinal ganglion cells (RGCs) and neurons of the scala tympani.

Activation of the target cells transmits sensory stimuli. The sensory stimuli may comprise visual stimuli, auditory stimuli, olfactory stimuli, mechanoreceptive stimuli, or nociceptive stimuli.

According to some embodiments, the electrodes can comprise a primary electrode and one or more secondary electrodes.

According to some embodiments, the spatial pattern can be derived from a linear model. The linear model can be used to determine how electrical signals from multiple electrodes will sum together to activate a cell.

### BRIEF DESCRIPTION OF THE DRAWINGS

FIG. 1 is a depiction of the experimental setup comprising the dense multi-electrode array.

FIGS. 2a-b are prophetic graphs showing how cells may be activated when two electrodes are in the region of three cells. FIG. 2a is a depiction of the region and a graph showing the spike probability when electrical signals are provided through each electrode. FIG. 2b is a graph showing iso-response contours of each cell.

FIG. 3a-b are prophetic graphs showing the probability that a cell may activate in response to electrical signal amplitudes passed through primary and secondary electrodes. FIG. 3a is a graph showing how electrical signals passing through a secondary electrode may affect the probability of a cell activating in response to electrical signals passed through a primary electrode. FIG. 3b is a graph showing an iso-response contour.

FIG. 4a-b are electrophysiological images showing the voltage signal generated on each electrode by a single cell when it fires an action potential.

FIG. 5a-b are graphs showing the responses to electrical stimulation. FIG. 5a are plots showing raw and artifact-subtracted voltage traces, and FIG. 5b are response curves.

FIG. 6 is a depiction of the estimated soma positions of five cell types in the primate retina, based on each cell's electrophysiological image.

FIG. 7a-b are graphs and a spatial sensitivity map showing the effects of applying electrical signals via a primary electrode and a secondary electrode spaced 30 μm apart. FIG. 7a are graphs showing the response probability and the iso-response contours of the cell when stimulated by pairs of electrodes comprised of the primary electrode and each of six secondary electrodes surrounding the primary electrode. FIG. 7b is a spatial sensitivity map of the secondary electrodes, depicting the modulatory effect of each secondary electrode on the probability of activating the cell.

FIG. 8a-b are graphs and a spatial sensitivity map showing the effects of applying electrical signals via a primary electrode and a secondary electrode spaced 60 μm apart. FIG. 8a are graphs showing the response probability and the iso-response contour of each of six secondary electrodes surrounding the primary electrode. FIG. 8b is a spatial sensitivity map of the secondary electrodes.

FIG. 9 are graphs that show the effects of applying electrical signals via a primary electrode and two secondary electrodes spaced 60 μm apart.

FIG. 10 are graphs that show the effects of applying electrical signals via a primary electrode and two secondary electrodes spaced 30 μm apart.

### DETAILED DESCRIPTION OF THE INVENTION

Sensory stimuli are complex signals that are recognized by the brain through the activation of specific patterns of sensory cells, which capture the different aspects of the signals. In order to mimic these patterns of activation, sensory neural prostheses selectively target and activate the sensory cells. Moreover, the neural prostheses, which is adapted to activate cells through the transmission of electrical signals, activates targeted cells without stimulating neighboring cells, as neighboring cells may carry different and sometimes contradictory sensory information.

For example, visual information is transmitted to the brain by different types of retinal ganglion cells (RGCs) that encode aspects of the visual stimulus and are spatially interspersed. Hence, visual prostheses electrically stimulate RGCs in specific patterns in order to reflect the natural activation of these cells by visual stimuli. Further, visual prostheses activate RGCs accurately, as activation of certain pathways, such as ON and OFF pathways (which differ by how light affects the firing rate of the cell), may have opposing interactions in the visual cortex. Therefore, sensory neural prostheses activate targeted cells without affecting neighboring cells in order to accurately transmit the sensory stimulus.

To this end, disclosed herein are embodiments directed toward activating sensory cells in a spatial pattern. Provided is a method, system, and algorithm for simultaneously providing electrical signals through multiple electrodes in a spatial pattern that maximally activates one or more target cells while minimizing activation of other cells. Such methods of targeted activation of sensory cells may be used in sensory neural prostheses.

Targeted sensory cells may be activated through application of electrical signals, such as current, to the cells. The electrical signals may be delivered to cells through an interface, such as electrodes. The electrodes may be part of a system, such as a device or prosthesis, in particular a sensory neural prosthesis, which controls the delivery of electrical signals to the cells. Examples of neural prostheses include, but are not limited to, visual prostheses such as epiretinal implants; auditory prostheses, such as cochlear implants, auditory brainstem implants, and auditory midbrain implants; prostheses for pain relief, such as a spinal cord stimulator; and sensory or motor prostheses. An example of an epiretinal implant is described in U.S. application Ser. No. 11/592,804, the entirety of which is incorporated herein by reference. Such a system could be adapted to implement embodiments in accord with this disclosure.

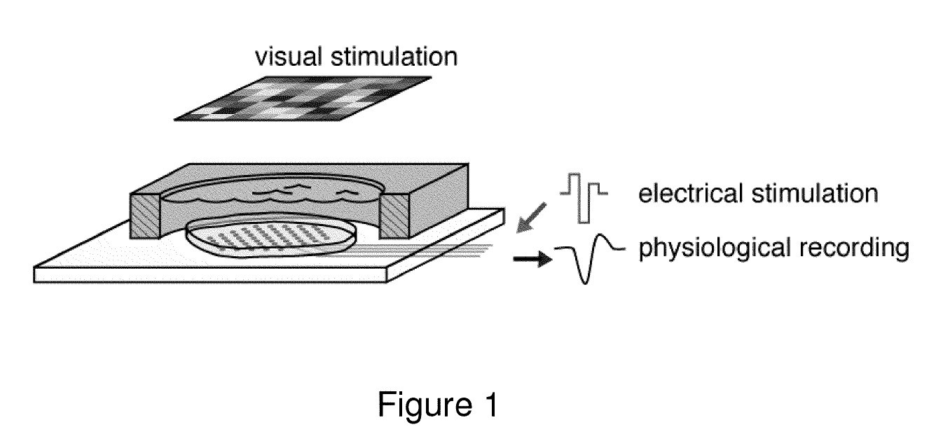

A simulation system used for in vitro applications in accord with one embodiment is shown in FIG. 1. The multi-electrode system independently and simultaneously addresses 61 electrodes for stimulation and recording, and uses platinum disk electrodes similar to those in human prosthetics but smaller and more densely packed by an order of magnitude, as was used in the examples and embodiments described herein. This system, as disclosed in the embodiments and examples herein, was used to apply stimulation patterns for selective activation and for producing spatial patterns of activity in populations of RGCs. The system injects current pulses through 61 independently-controlled 5-15 μm diameter platinum electrodes with 30 or 60 μm inter-electrode spacing. This stimulation system includes circuitry to reduce electrical artifacts, permitting detection of low-latency (<1 ms) RGC responses on all electrodes including the electrode that is used for stimulation (see Hottowy et al. An integrated multichannel waveform generator for large-scale spatio-temporal stimulation of neural tissue. Analog Integrated Circuits and Signal Processing, 2008, 55: 239-248; see also Sekirniak et al. High resolution electrical stimulation of primate retina for epiretinal implant design. Journal of Neuroscience, 2008, 28: 4446-4456; the entirety of which are both incorporated herein by reference).

The electrodes may interface a population of cells, such as sensory cells. Examples of sensory cells may include, but are not limited to, RGCs and neurons of the scala tympani.

Target cells are identified according to the different aspects of sensory stimuli. For example, which RGCs in a retina are targeted will depend on the different spatial patterns and color of light entering the visual field.

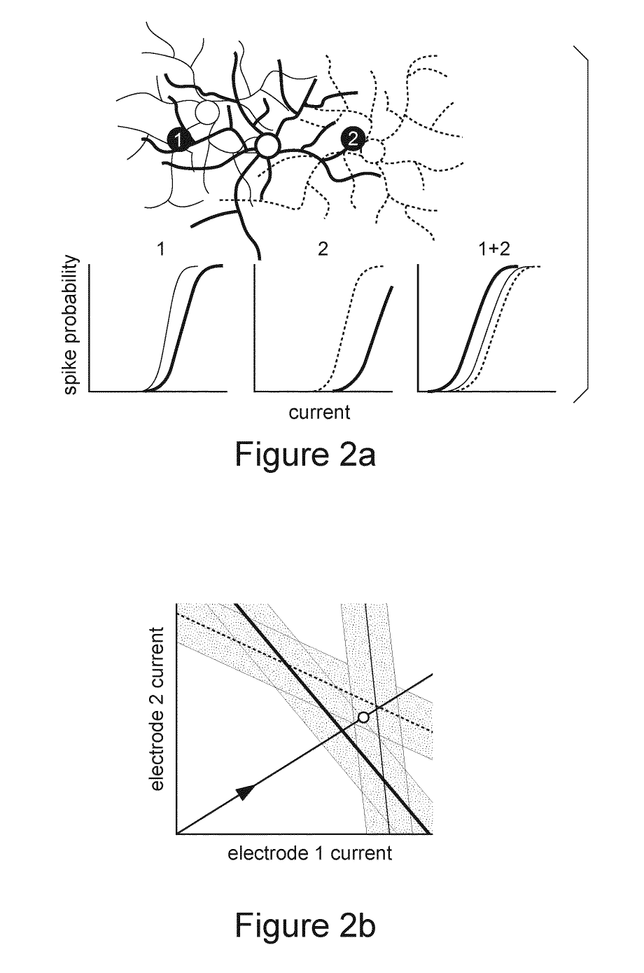

A stimulation pattern for selectively activating sensory cells can be achieved using two or more electrodes, i.e., a primary electrode and one or more secondary electrodes. The primary electrode is the electrode to which the cell is maximally sensitive, while the secondary electrodes are the surrounding electrodes. Simultaneous application of an electrical stimulus through a secondary electrode can change the amount of current on the primary electrode required to activate the cell, as depicted by linear equation (1) and shown in FIG. 2a-b

FIG. 2a is a graph prophetically demonstrating how three cells, i.e., a blue cell, a purple cell, and a red cell, may be activated by a pair of electrodes. Application of electric signals by electrode 1, which is close to the blue cell, may lead to activation of the blue cell at a lower signal amplitude than the other cells. Similarly, application of electric signals by electrode 2, which is close to the purple cell, may lead to activation of the purple cell at a lower signal amplitude than the other cells. However, to activate the red cell without activating either of the other two cells, both electrodes can apply electric signals simultaneously, which under certain conditions lowers the threshold of the red cell sufficiently to activate the red cell first.

This is also prophetically depicted in the iso-response contours of each cell in FIG. 2b. Application of electric signals through electrode 1 alone may be equivalent to moving right along the x-axis. As the amplitude of the electric signal increases along the x-axis, the blue cell may begin to respond (as marked by the inner edge of the blue shaded region). The signal may reach an amplitude at which the threshold is 50% (as marked by the blue line), and eventually an amplitude wherein the blue cell responds nearly all of the time (as marked by the outer edge of the blue region). Similarly, electrical signals from electrode 2 alone may be equivalent to move up along the y-axis, and the amplitude may affect the activation of the purple cell. However, as shown by the red shaded region and red line, electrode 1 and electrode 2 may apply electrical signals at particular amplitudes whereby only the red cell may activate. Further, the red cell may be activated at a lower electrical signal than either of the other cells. Thus, selectivity of target cells using multi-electrode stimulation may be achieved by finding the particular combination of the electrical signals on the electrodes that selectively activates the target cell.

Notably, reversing polarity of stimulation on the secondary electrode may have an equal and opposite effect on the primary current threshold.

The probability of a cell activating in response to electrical signals passed through a primary electrode and two or more secondary electrodes can be modeled using a linear equation (1):

P=f(I₀+λ₁I₁+λ₂I₂+ . . . +λₙIₙ) (1);

wherein Iᵢ is the current passed through the i^{th} electrode, and λᵢ is the sensitivity of the cell to the i^{th} secondary electrode. I₀, the current passed through the primary electrode, can be represented by equation (2):

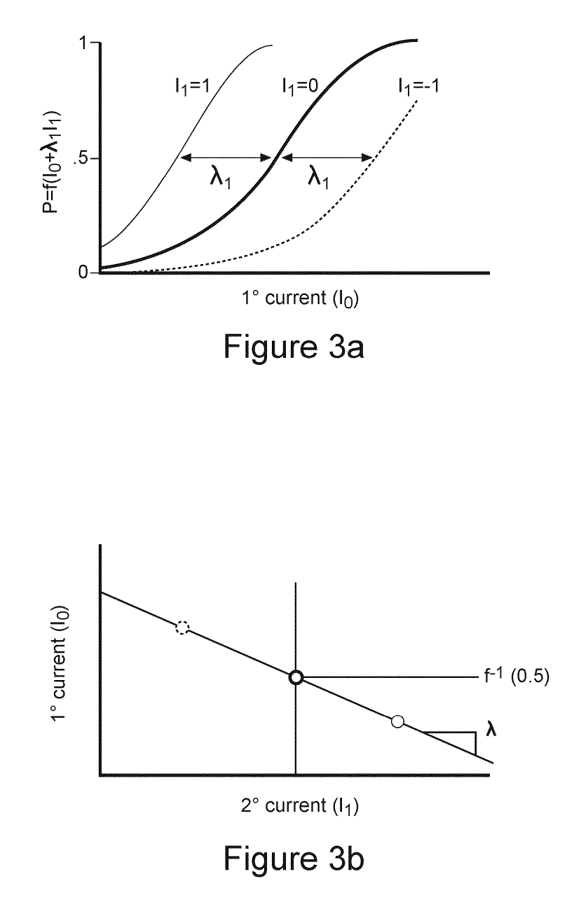

I₀=f⁻¹(0.5)−λ₁I₁−λ₂I₂− . . . −λₙIₙ (2);

wherein f⁻¹(0.5) is the activation threshold using the primary electrode alone. This linear model is depicted in FIG. 3a-b. In FIG. 3a, stimulating with the primary electrode alone may result in the blue response curve; simultaneously stimulating with 1 microamp current of the same polarity on the secondary electrode may lead to a shift of the response curve by lambda in one direction; injecting opposite polarity current may lead to a shift in the opposite direction by the same amount; and injecting ½ as much current may lead to ½ the shift. FIG. 3b depicts an iso-response contour showing the threshold of the cell. The input electrical signals may add linearly, which is reflected by the fact that this contour is in fact a straight line.

In general, pattern of activation in response to an electrical signal can be predicted using equation (3):

r=T s (3);

wherein s is a column vector containing real-valued entries representing the current passed through a collection of M electrodes; r is a column vector containing 0's and 1's for a collection of N cells, indicating whether each cell was activated or not in a particular time window after electrical stimulation; and T is a N×M matrix transformation in which each row represents the sensitivity of a single cell to stimulation of the various electrodes. In general, each row of T contains several nonzero entries (meaning that a given cell responds to more than one electrode), and each column of T also contains several nonzero entries (meaning that a given electrode contributes to activation of several cells). Since a given electrode does contribute to the activation of several cells, electrical stimulation through a single electrode in general cannot activate just one cell.

Selective activation of one cell can be achieved, as shown when equation (3) is rewritten as equation (4): because above expression can be rewritten

s=T⁻¹ r (4)

wherein T⁻¹ is the inverse of the matrix T. The pattern of stimulation s required to stimulate a single cell can be determined using equation (4). For example, if only the first cell is stimulated, r would be a vector wherein the first entry is 1 and the remaining entries are zero. Applying this r to equation (4) results in s as a vector that activates only the first cell.

The values of r may represent not just activation or failure, but the probability of activation averaged over many repeated attempts, in which case the entries are real numbers between 0 and 1.

The probability of activation may be related in a direct, but nonlinear, way to the matrix product in equation (3), which can be accommodated in this model. For example, if function q( ) is a nonlinear function of one variable that takes values between 0 and 1, such q(x)=erq(x), where erq( ) is the sigmoidal (cumulative normal) function, then equations (3) and (4) can be replaced with equations (5) and (6), respectively:

k=T s (5);

s=T⁻¹ k (6);

and then,

rᵢ=q(kᵢ) (7);

wherein rᵢ and kᵢ are the i^{th} elements of r and k, respectively. In this case, the solution for selective activation is obtained by choosing a desired response vector r with a single nonzero entry, then computing the relevant value of k by using the inverse function on each element of r:

kᵢ=q⁻¹(rᵢ) (8);

The stimulus s is computed from k using equation (6).

Similarly, a nonlinear function applied to the stimulus prior to its linear combination can be considered, that is,

pᵢ=g(sᵢ) (9)

and

r=T p (10)

In this case, for a given desired stimulation pattern r, p is computed using equation (11):

p=T⁻¹ r (11);

and then the necessary stimulus is computed using equation (12):

sᵢ=g⁻¹(pᵢ) (12).

Both forms of nonlinearity can be used in combination, as shown in equations (13)-(15:

k=Tp (13);

pᵢ=g(sᵢ) (14);

rᵢ=q(kᵢ) (15).

Although the above refers to selective activation of a single neuron (one entry of r is nonzero), in general this framework permits the calculation of the pattern of stimulation required to produce any pattern of activity in the population. For any desired pattern of activity r, the stimulus required to elicit that pattern is computed using the above logic.

Measurement of the above transformation T can be accomplished by providing different current levels on each electrode (i.e. different values of s), measuring the activation pattern (values of r) and performing a linear regression of r against s. However, for prosthetic devices, T may be estimated rapidly without electrical stimulation using a more direct means. For instance, if the firing of the i^{th} neuron produces a pattern of electrical activity wᵢ on the different electrodes (w is a column vector of length M, the number of electrodes), when the electrodes are used for measuring electrical activity rather than stimulation, then the assumption can be made that the probability of activation of a given cell is proportional to the electrical potential created by the firing of that cell on the electrode. Thus, the i^{th} row of T can be approximated by the vector wᵢ.

### EXAMPLES

### Example 1

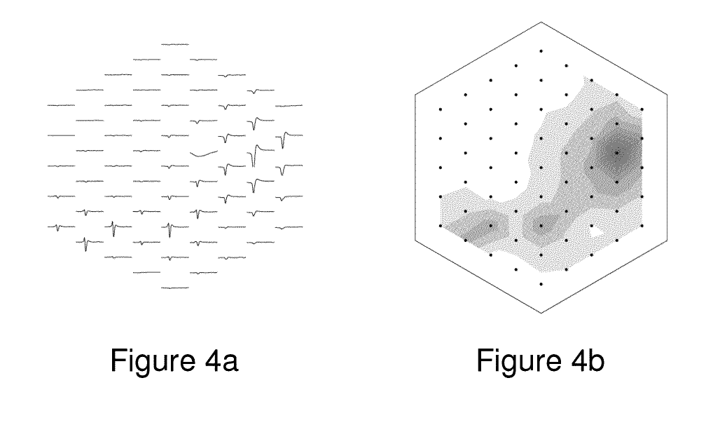

The system depicted in FIG. 1 can be used to stimulate RGCs. A small segment of retina is placed RGC-side down on the array of electrodes. FIG. 4a-b shows an electrophysiological image of the voltage signal generated on each electrode by a spike from the cell. The average voltage signal of over thousands of spikes can be used to calculate the contours depicting the approximate location of the cell, as shown in FIG. 4b. The dark shades of blue shown in FIG. 4b denotes a greater amplitude of average voltage signal.

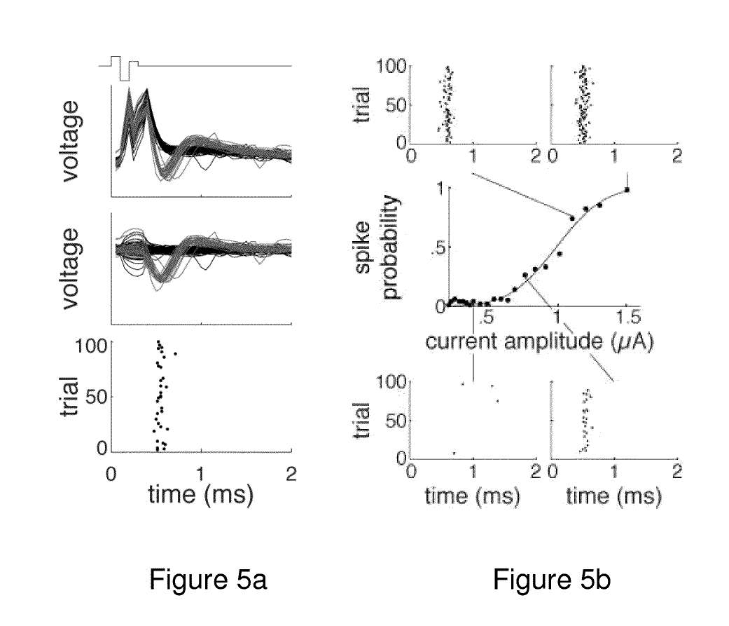

Responses to electrical stimulations were determined by passing current with a biphasic or triphasic charge-balanced waveform with a duration of about 0.1 ms and an amplitude of about 1 μA. Waveforms with spikes were separated from waveforms with only the stimulus artifact. Current was varied while examining the spike response from a nearby electrode or the same electrode. FIG. 5a-b shows the electrical activation of an ON-midget RGC by 100 applications of a low-amplitude (0.91 μA, 50 μs cathodic phase) electrical pulse. The top plot of FIG. 5a shows voltage traces recorded on the stimulating electrode, comprising electrical artifact (black line) and combined artifact and spike (red). The middle plot of FIG. 5a shows the result of subtracting the average artifact-alone trace from both groups, which revealed a spike wave form that can determine the identity of the cell (in this case, an ON-midget RGC). The bottom plot of FIG. 5a shows the times of evoked spikes on each trial. FIG. 5b shows the spike probability as a function of current amplitude, and the times of evoked spikes on each trial at different current amplitudes. The spike probability revealed a 50% activation threshold of roughly 1 μA.

### Example 2

To observe the spatial intermixing of many distinct RGC types that transmit different information to different regions of the brain, in vitro multi-electrode array recordings were taken of the primate retina. The soma locations of nearly all of the RGCs in the five highest-density cell types were estimated from the voltage signal generated at each electrode when each cell spikes.

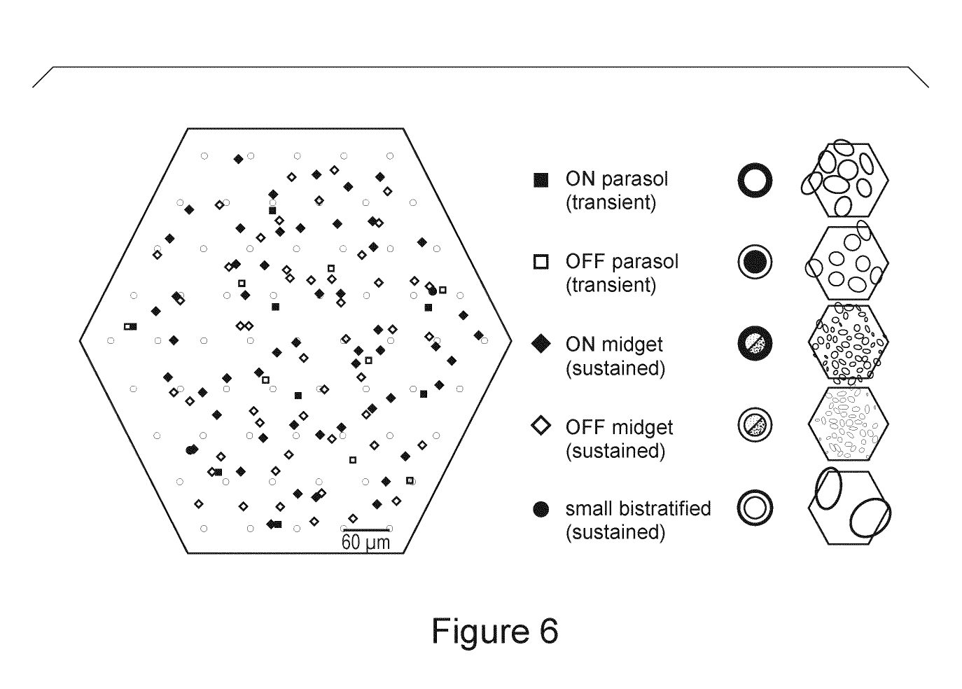

As shown in FIG. 6, distinct RGC types were highly intermixed. Notably, an electrode of a 100 μm diameter, which is comparable to electrode sizes used in current epiretinal prostheses, activated cells of many different types simultaneously.

### Example 3

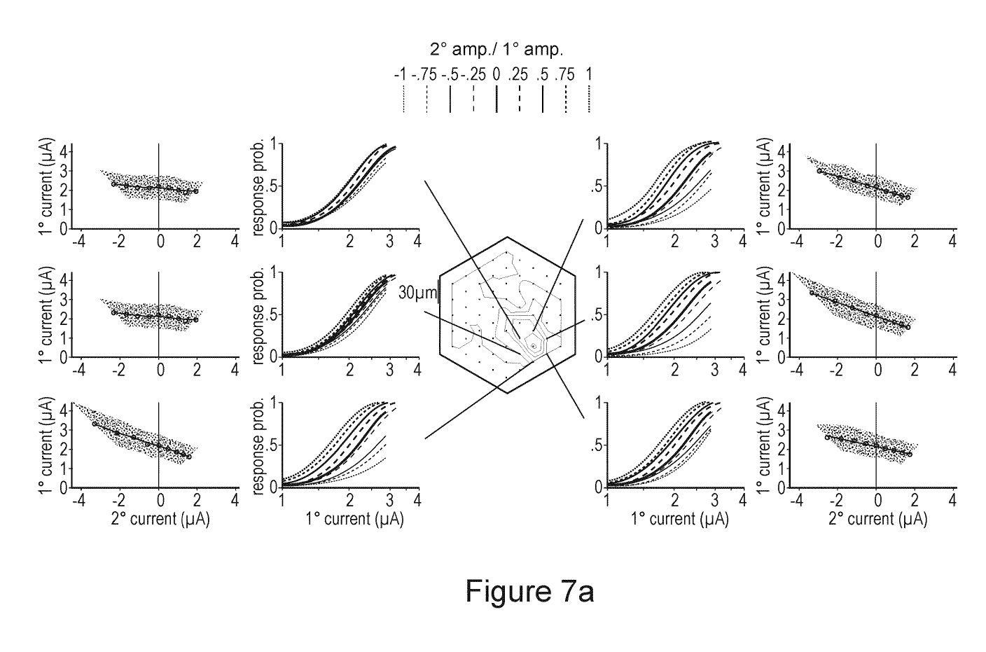

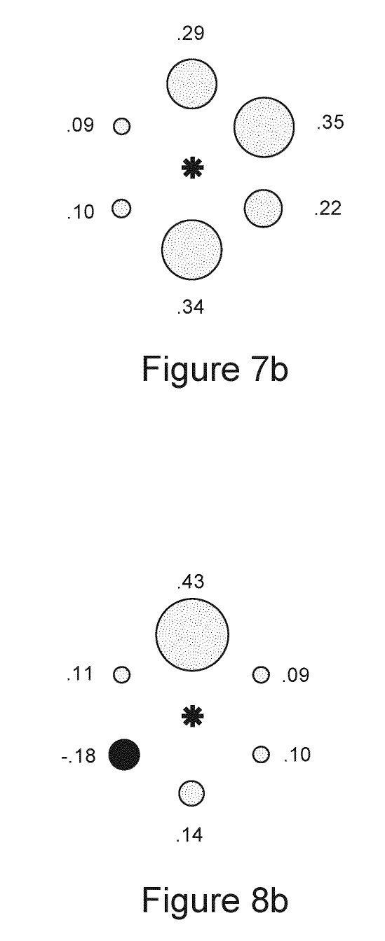

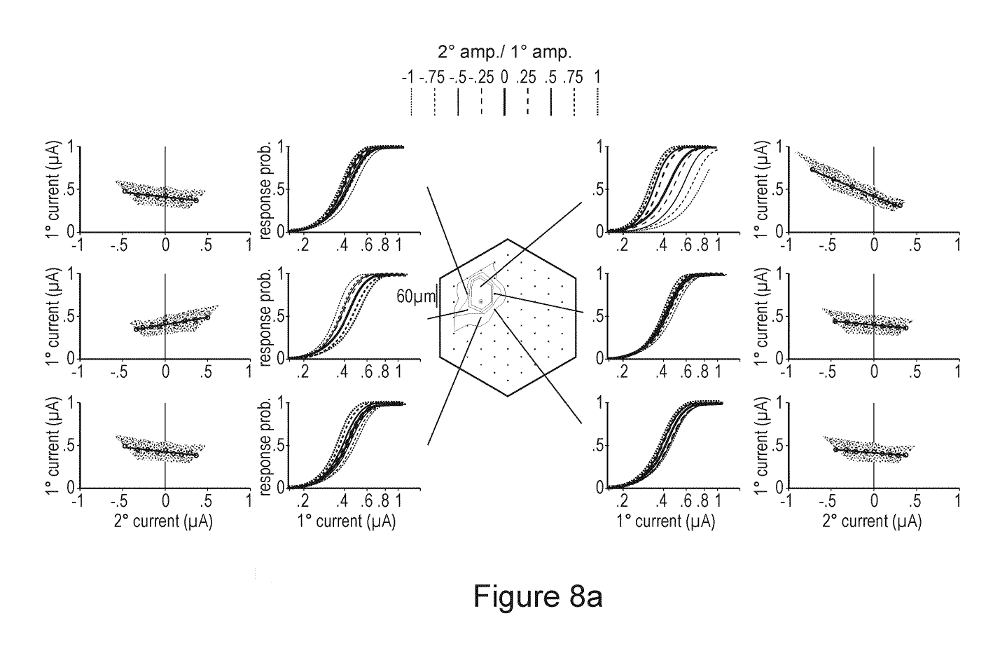

In order to test the linear model of the present invention, the primary electrode for a given cell was first identified, and then the effects of applying electrical signals through each of the surrounding “secondary” electrodes at different amplitudes and polarities were determined. The results for 2 cells, one stimulated with an array with 30-micron electrode spacing and the other with 60-micron electrode spacing, are shown in FIGS. 7a-b and 8a-b, respectively. FIGS. 7a and 8a show the response probability (left plot of each pair of graphs) and the iso-response contour (right plot of each pair of graphs) resulting from stimulation using the primary electrode and one of the secondary electrodes, at varying stimulation amplitude ratios. In the map showing the location of the electrodes shown in the middle of FIGS. 7a and 8a, the primary electrode is indicated by a white asterisk and location of the voltage signal generated by the cell's spikes (the electrophysiological image) is indicated by the blue shading.

In the response probability plots, the response to stimulation with the primary electrode alone is given by the black curve, while the responses to same-polarity stimulation on the secondary electrode with different amounts of current are shown as blue curves; lighter blue indicates higher amplitude secondary currents. The responses to opposite polarity secondary electrode stimulation are shown by the red curves, with lighter red indicating higher amplitude opposite-polarity secondary currents. The response probability plots show that same-polarity stimulation moved the response curves to the left, thereby lowering the threshold, and that the size of the shift increased as the amount of secondary current increased. As expected from the linear model, opposite polarity secondary stimulation caused a shift in the opposite direction, increasing the threshold in proportion to the amplitude of the secondary current.

The iso-response plots of FIGS. 7a and 8a shows that the threshold varies linearly with the current injected through the secondary electrode. In these plots, opposite-polarity stimulation is indicated by a negative current value.

When electrodes are spaced at 30-micron apart, as shown in FIG. 7a, the linear model applied for all of the secondary electrodes, and in every case, the same-polarity stimulation on the secondary electrode lowered threshold while opposite-polarity current increased threshold. The size of the effect varied considerably with the secondary electrode, as some secondary electrodes had little effect (e.g., top and middle sets of the left column of plots) while some had a strong effect (e.g., lower set of the left column of plots and the top and middle set of the right column of plots). The different effects of the different secondary electrodes may be summarized by determining the slope of each secondary electrode in the iso-response plots, which correspond to the lambda values in the linear model of equation (1), and plotting these values as circles with diameters proportional to the lambda values at the position of the electrodes as shown in FIG. 7b.

In FIG. 8a-b, which depicts a cell stimulated/recorded by an array with 60 micron spacing, the modulatory effect was smaller for most secondary electrodes, likely due to the greater distance from the primary electrode. Also, in one case (middle set of left column of plots), the secondary electrode had the opposite effect in terms of the direction of the threshold shift, such that when the same-polarity current was passed through this secondary electrode, the threshold increased rather than decreased, and opposite-polarity current decreased rather than increased threshold. This is still consistent with the linear model and results in a negative lamda value, which is depicted in the spatial sensitivity map as a red circle (see FIG. 8b).

### Example 4

The linear model as depicted in equation (1) also predicts that the shifts induced by individual secondary electrodes will sum when used together. This was tested by measuring the threshold shifts due to individual secondary electrodes, and then measuring the threshold shift when the stimulation pattern consisted of the primary electrode and a neighboring pair of secondary electrodes.

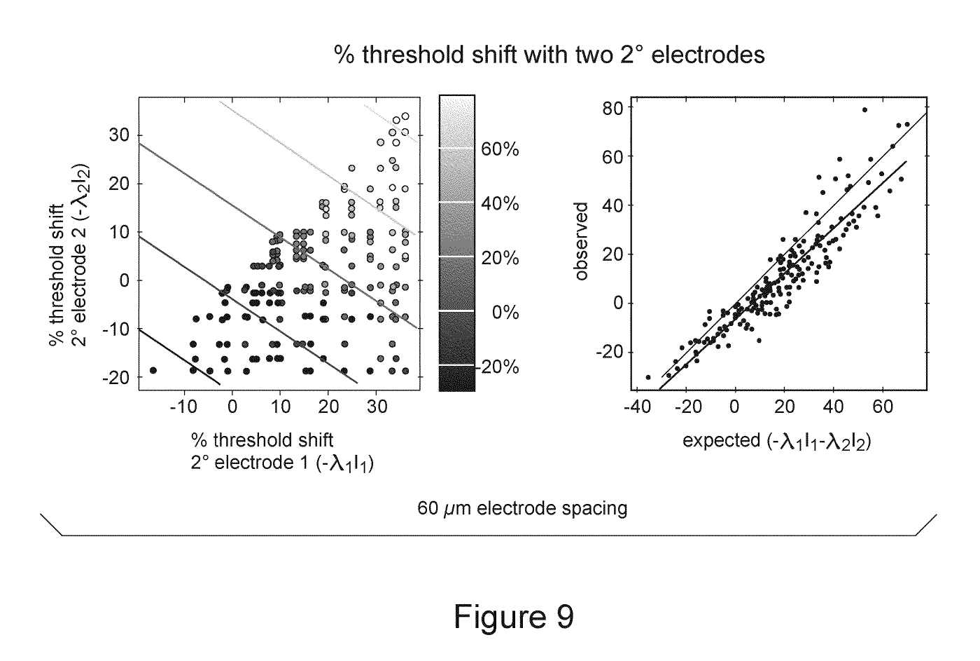

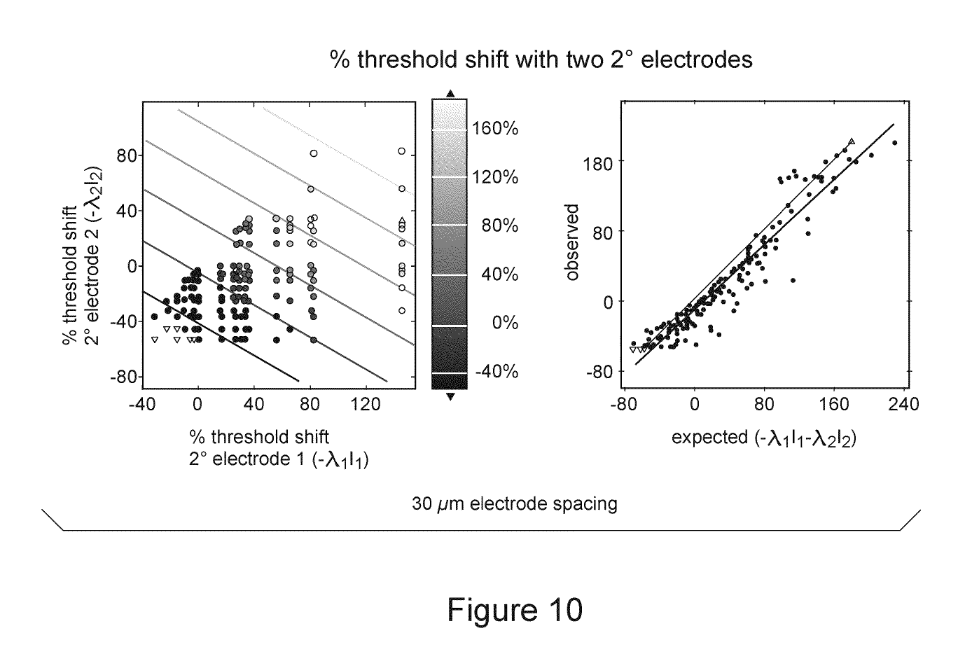

The predicted vs. observed results are shown in FIGS. 9 and 10, wherein the electrodes are distributed at 60 μm and 30 μm spacing, respectively. The left plot of each figure depicts how the probability response threshold shifted when the secondary electrodes applied electrical signals simultaneously with the primary electrode, as compared to when a primary electrode applied electrical signals alone. The shifts resulting from pairs of secondary electrodes are plotted as colored dots that indicate the observed shift in threshold. The prediction of the model is shown by the fact that the colors increased linearly from the bottom left corner to the top right corner.

The right plot of each figure compares predicted vs. observed results. The data points lie close to the line, which indicates that the effects of individual secondary electrodes were roughly additive. Hence, measurement of the sensitivity parameter lambda for each secondary electrode can be used to predict the response to any combination of amplitudes on the primary electrode and the six neighboring secondary electrodes.

Accordingly, while the invention has been described and illustrated in connection with preferred embodiments, many variations and modifications as will be evident to those skilled in this art may be made without departing from the scope of the invention, and the invention is thus not to be limited to the precise details of methodology or construction set forth above, as such variations and modification are intended to be included within the scope of the invention. Therefore, the scope of the appended claims should not be limited to the description and illustrations of the embodiments contained herein.

What is claimed is:

1. A method comprising:
interfacing a population of cells with two or more electrodes;
identifying target cells within the population to activate by electrical signals; and
applying electrical signals in a spatial pattern to the population via the electrodes, wherein the target cells are activated.

2. The method of claim 1, wherein the population of cells comprise sensory cells.

3. The method of claim 2, wherein the sensory cells are retinal ganglion cells.

4. The method of claim 2, wherein the sensory cells are neurons of the scala tympani.

5. The method of claim 1, wherein activation of the target cells transmits sensory stimuli.

6. The method of claim 5, wherein the sensory stimuli comprise visual stimuli, auditory stimuli, olfactory stimuli, mechanoreceptive stimuli, or nociceptive stimuli.

7. The method of claim 1, wherein the electrodes comprise a primary electrode and one or more secondary electrodes.

8. The method of claim 1, wherein the spatial pattern is derived from a linear model.

9. The method of claim 8, wherein the linear model determines how electrical signals from multiple electrodes will sum together to activate a cell.

10. The method of claim 8, wherein the electrodes comprise a primary electrode and one or more secondary electrodes.

11. The method of claim 10, wherein the probability of a cell activating in response to electrical signals passed through a primary electrode and two or more secondary electrodes is modeled using a linear equation (1):
P=f(I₀+λ₁I₁+λ₂I₂+ . . . +λₙIₙ)
wherein Iᵢ is the current passed through the i^{th} electrode, and λᵢ is the sensitivity of the cell to the i^{th} secondary electrode.

12. The method of claim 11 wherein the current passed through the primary electrode I₀ is represented by equation (2):
I₀=f⁻¹(0.5)−λ₁I₁−λ₂I₂− . . . −λₙIₙ
wherein f⁻¹(0.5) is the activation threshold using the primary electrode alone.

13. The method of claim 7, wherein the electrodes are configured for a sensory neural prosthesis for a subject.

14. An electrode array for a sensory neural prosthesis comprising:
two or more electrodes;
wherein the array is configured to interface with a population of sensory cells of a subject and apply electrical signals in a spatial pattern to the population via the electrodes, wherein target cells within the population are activated by electrical signals from the array.

15. The array of claim 14, wherein the sensory cells are retinal ganglion cells.

16. The array of claim 14, wherein the sensory cells are neurons of the scala tympani.

17. The array of claim 14, wherein activation of the target cells transmits sensory stimuli.

18. The array of claim 17, wherein the sensory stimuli comprise visual stimuli, auditory stimuli, olfactory stimuli, mechanoreceptive stimuli, or nociceptive stimuli.

19. The array of claim 14, wherein the electrodes comprise a primary electrode and one or more secondary electrodes.

20. The array of claim 14, wherein the spatial pattern is derived from a linear model.

21. The array of claim 20, wherein the linear model determines how electrical signals from multiple electrodes will sum together to activate a cell.
